(19) United States
(12) Patent Application Publication
(10) Pub. No.: US 2004/0131637 A1
(43) Pub. Date: Jul. 8, 2004

(54) Salmonella promoter for heterologous gene expression

(76) Inventor: Steven Neville Chatfield, Berkshire (GB)

Correspondence Address: Saliwanchik Lloyd & Saliwanchik, Suite A 1, 2421 NW 41st Street, Gainesville, FL, 32606-6669, US

(21) Appl. No.: 10/471,385

(22) PCT Filed: Mar. 11, 2002
(86) PCT No.: PCT/GB02/01098

(30) Foreign Application Priority Data
Mar. 9, 2001 (GB) 0105924.5

(51) Int. Cl. A61K 39/02; C12N 1/21; C12N 15/74

(52) U.S. Cl. 424/200.1; 435/252.3; 435/471; 435/320.1

(57) ABSTRACT

The ssaG derived from Salmonella is shown to exert improved expression of heterologous genes compared to other known promoters and therefore can be used advantageously in constructs for the delivery of therapeutic proteins to a patient.

### FIELD OF THE INVENTION

This invention relates to promoter sequences which promote the expression of a suitable polynucleotide.

### BACKGROUND OF THE INVENTION

There is now widespread interest in the use of attenuated microorganisms as vaccines. It is also proposed that attenuated microorganisms may be useful for the delivery of therapeutic agents.

The ssaG gene is a component of the Salmonella pathogenicity island SPI-2 (WO96/17951).

Valdivia et al., Science, 1997; 277 (5334): 2007-2011 describes a promoter trap experiment to identify bacterial genes that are preferentially expressed in a host cell. The ssaH gene (now known as ssaG) is identified as a gene that is expressed when Salmonella typhimurium infects a host's macrophage cells. It is disclosed that the ssaH (ssaG) promoter is fused with the gene for green fluorescent protein (GFP) and placed on a multicopy plasmid system to establish whether expression occurs, thereby determining whether the ssaH gene would be expressed on infection.

### SUMMARY OF THE INVENTION

According to a first aspect of the invention, a construct comprises the ssaG promoter, or a functional fragment thereof, operably linked to a polynucleotide heterologous to the ssaG gene.

According to a second aspect of the invention, a Salmonella microorganism comprises a heterologous polynucleotide operably linked to the ssaG promoter.

According to a third aspect, the ssaG promoter is used to promote expression of a polynucleotide heterologous to that of the ssaG gene and which encodes a therapeutic protein or peptide.

According to a fourth aspect, a method for the expression of a heterologous polynucleotide within a Salmonella microorganism, comprises integrating the polynucleotide into the microorganism's chromosome so that it is operably linked to the endogenous ssaG promoter.

### DESCRIPTION OF THE DRAWINGS

The invention is illustrated with reference to the accompanying drawings where:

FIG. 1 is a schematic representation of ssaG promoter regions cloned into S. typhimurium strains and GFP reporter vectors, wherein the arrows indicate the regions of S. typhimurium TML DNA derived from upstream of the ssaG ATG start codon;

FIG. 2 shows the level of LT-B expression in S. typhimurium strains harbouring different regions of the ssaG promoter inside macrophages;

FIG. 3 shows the serum IgG anti-LT-B responses in BALB/c mice on day 28 post-immunisation with various strains (Study 1);

FIGS. 4a and 4b show respectively the serum IgG anti-LT-B responses in BALB/c mice on days 28 and 42 post-immunisation (Study 2);

FIGS. 5a and 5b show respectively the serum IgG anti-LT-B responses in BALB/c mice on days 28 and 42 post-immunisation (study 3);

FIG. 6 shows a FACS analysis of GFP expression in S. typhimurium strains infecting J774A-1 cells;

FIG. 7 shows GFP expression in S. typhimurium strains infecting J774A-1 cells, taken at points up to 24 hours; and

FIG. 8 shows LacZ expression in S. typhimurium strains harbouring GFP/LacZ reporter vector infecting J774A-1 cells, taken at points up to 24 hours.

### DESCRIPTION OF THE INVENTION

The present invention is based on the discovery that the ssaG promoter is surprisingly effective at promoting the expression of heterologous polynucleotides. The promoter may therefore be used in therapy to drive expression of polynucleotides encoding therapeutic proteins or peptides etc. It should be understood that references to therapy also include preventative treatments, e.g. vaccination. Furthermore, veterinary applications are also considered to be within the scope of the invention.

The ssaG promoter is located upstream of the start codon for the ssaG gene, identified in Hensel et al. Mol. Microbiol., 1998; 30(1): 163-174. The sequence identified herein as SEQ ID NO. 1 contains the functional promoter and may be used as part of the invention. Functional fragments of this sequence, including fragments with high identity, are also within the scope of the invention. The sequence identified herein may be further fragmented to obtain more defined polynucleotides comprising the promoter sequence. Synthetic or recombinant techniques may be used to generate the shorter fragments which retain the promoter function. The fragment may comprise at least 30 nucleotides, preferably at least 40 nucleotides and most preferably at least 60 nucleotides. Sequence identity may be at least 50%, preferably 60% and most preferably at least 90%.

Preferably, the promoter comprises at least the sequence from nucleotide number 330 to 503 (173 bp) of SEQ ID NO. 1, more preferably at least the sequence from nucleotide number 229 to 503 (275 bp) and most preferably from nucleotide number 39 to 503 (465 bp) of SEQ ID NO. 1.

The term “identity” is known in the art. The use of the term refers to a sequence comparison based on identical matches between correspondingly identical positions in the sequences being compared.

Levels of identity between gene sequences can be calculated using known methods. In relation to the present invention, publicly available computer-based methods for determining identity include the BLASTP, BLASTN and FASTA (Atschul et al, J. Molec. Biol., 1990; 215:403-410), the BLASTX programme available from NCBI, and the Gap programme from Genetics Computer Group, Madison Wis. The levels of identity may be obtained using the Gap programme, with a Gap penalty of 50 and a Gap length penalty of 3 for the polynucleotide sequence comparisons.

The promoter may be isolated and used as part of a recombinant construct or vector, for delivery into a host cell etc. Alternatively, in the context of Salmonella microorganisms, the endogenous promoter may be used to drive expression of a heterologous polynucleotide (or gene) which is inserted downstream of the promoter.

The skilled person will appreciate that recombinant DNA techniques can be used to produce the constructs and recombinant microorganisms according to the invention.

A construct according to the invention may be in the form of an expression vector or plasmid, comprising the promoter, operably linked to the heterologous polynucleotide. Additional selection marker genes or regulatory elements may also be included as part of the construct. The construct may also be designed so that it is capable of integrating within a host's chromosome, for example by the utilisation of transposable elements.

If the promoter is to be used endogenously, with a heterologous polynucleotide inserted functionally downstream, then various techniques may be used to achieve this, including homologous recombination. All this will be apparent to the skilled person.

The polynucleotide for use in the invention may be any that encodes a product that has a therapeutic utility. Therapeutic products are those that are useful in the treatment or prevention of a condition or disease of the human or animal body. For example, the polynucleotide may encode a protein that acts at a particular receptor site. Hormones and growth factors are therefore within the scope of the invention. Alternatively, the encoded product may be antigenic, eliciting an immune response. Typically, the antigenic fragments (product) will be at least 10 amino acids in size, preferably at least 20 amino acids and most preferably at least 30 amino acids in size.

Suitable Antigens Include:

Allergy Vaccines

B cell mIgE peptide (Tanox),

human IgE decapeptide (PT),

allergen peptides e.g. cat dander (feI d), house dust mite (Der p 1, Der f 1), and companion animal vaccines e.g. canine IgE peptides

Cancer Vaccines

MUC1 (human mucin expressed by breast (and other) epithelial cancer cells,

HER-2/neu peptides expressed by breast cancer cells,

EGFRvIII (Variant of epidermal growth factor receptor expressed on numerous cancer cell types),

hCG peptides (Keutmann loop and CTP-37) expressed by bladder cancer cells,

idiotypic peptides expressed by human lymphoma cells,

P53 peptides,

Ras peptides,

MAGE, BAGE, GAGE, tyrosinase, and CTL epitopes etc. for melanoma;

Fertility Vaccines

hCG peptides (Keutmann loop and CTP-37),

Peptides from sperm antigens (e.g. FA-1 and FA-2, lactate

dehydrogenase, SP-10, NZ-1, NZ-2),

Peptides from oocyte antigens (e.g. Zonula pellucida-3)

Viral Vaccines

Respiratory syncitial virus_(RSV) (F and G proteins),

Measles virus (MV) (F protein),

HIV proteins/peptides: (gp41 Kennedy epitope), gp 120 major

immunodominant loop (concensus sequence) chemokine receptor

peptides e.g. CXCR4, CCR5; nef, rev, pol, tat,

Canine/mind Parvovirus (VP1 peptide),

Hepatitis A virus (VP1),

Hepatitis B virus (HbcAg, HbcAg/Pre-S1, HbsAg, S-loop peptide),

Human papilloma virus L1, L2, E2, E6 and E7,

Hepatitis C virus (E1 and E2 HVR 1 peptides), core antigen,

HSV-1 gpD,

HSV-2 gpG,

Rotavirus VP7, VP6, VP4,

Parasite Vaccines

Malarial: (CSP (NANP)n, SSP-2, MSP-1, AMA-1, RAP-1, EBA 175, LSA-1),

Schistosome (GST, TPI, GAPDH, paramyosin)

Fungal Vaccines

C. albicans proteins/peptides

Bacterial Vaccines

P. aeruginosa proteins (OmpF and OmpI),

S. aureus proteins (FnBP),

S. epidermidis proteins (FiBP),

Chlamydia proteins,

EPEC/EHEC, e.g. intimim,

ETEC (CFA, LT-B),

Pertussis (pertactin, FHA),

Tetanus (TffC),

Non-typeable H. influenzae (P6 protein),

Others

Cytokine Genes, Growth Factors

The heterologous polynucleotide may be a therapeutic nucleic acid, e.g. one that is transcribed to produce an anti-sense RNA.

The constructs of the invention may be used in any suitable microorganism for the delivery of the therapeutic protein, peptide, RNA etc. In a preferred embodiment, the microorganism is a Salmonella microorganism, e.g. S. typhi or S. typhimurium. However, other gram-negative microorganisms, e.g. E. coli and Shigella can also be used as the delivery vehicle by the incorporation of the ssaG promoter and heterologous gene.

The microorganism will usually be attenuated, i.e. of reduced virulence. The attenuation of virulence is known to those skilled in the art and methods for preparing such microorganisms are well known. Virulence genes are known, and, in the context of Salmonella, include those located within SPI-2. The genes may be targetted for inactivation with the insertion of ssaG promoter:heterologous gene fusion. A particularly preferred attenuated microorganism is a Salmonella strain that is mutated to prevent expression of the ssaV and aroC genes. Salmonella strains disrupted in this way are disclosed in WO00/68261. The promoter:gene construct may be inserted within one of the disrupted genes, e.g. aroC.

In a further preferred embodiment, the heterologous polynucleotide is inserted functionally downstream of the ssaG promoter in an attenuated Salmonella microorganism. The insertion of the heterologous polynucleotide may disrupt the ssaG gene, which may in turn result in attenuation. The attenuated microorganism may then be used in a vaccine preparation, with the heterologous polynucleotide further promoting the prophylactic effect. Vaccine compositions can be formulated with suitable carriers or adjuvants, e.g. alum, as necessary or desired, to provide effective immunisation against infection.

The ssaG promoter is also shown in the experiments detailed below to be highly regulated with expression limited to conditions occurring in the natural macrophage environment. This may offer advantages for the controlled expression of genes encoding toxic (e.g. cytotoxic) products. The promoter can therefore be used to express proteins in specific environments in vivo or in vitro. In addition, specific induction of the promoter activity may be useful for the production of proteins etc. in a fermentation process. All this will be apparent to the skilled person.

The following Example illustrates the invention.

### EXAMPLE

A series of Salmonella typhimurium mutant strains were constructed to test the effectiveness of the ssaG promoter in gene expression. The strains were then tested in cell culture, in vitro and in vivo experimental models to study gene expression.

The ssaG promoter was compared to an alternate in vivo inducible pagC promoter. This promoter drives expression of the pagC gene, which encodes a 188 amino acid outer membrane protein required for full virulence of S. typhimurium in mice (Pulkkinen and Miller, J. Bacteriol., 1991: 173(1): 86-93). pagC is a member of the PhoP/PhoQ regulon and is upregulated 77-fold in S. typhimurium infected mouse macrophages harbouring a multicopy pagC-lacZ reporter vector (Alpuche-Aranda et al., PNAS, 1992; 89(21): 10079-83). The pagC promoter has been investigated in several studies as an in vivo inducible promoter for the delivery of foreign antigens by S. typhimurium. Dunstan et al., Infect. Immun., 1999; 67(10): 5133-41, reported that the pagC promoter functioned more effectively than the nirB and katG in vivo inducible promoters for the delivery of antigens using a multicopy lacZ/luciferase reporter vector in S. typhimurium ΔaroAD strains. Also, the pagC promoter, integrated as a single chromosomal copy, has also been shown to enhance the immunogenicity of model antigens in mice in comparison to constitutive promoters (Hohmann et al., PNAS, 1995; 92(7): 2904-8).

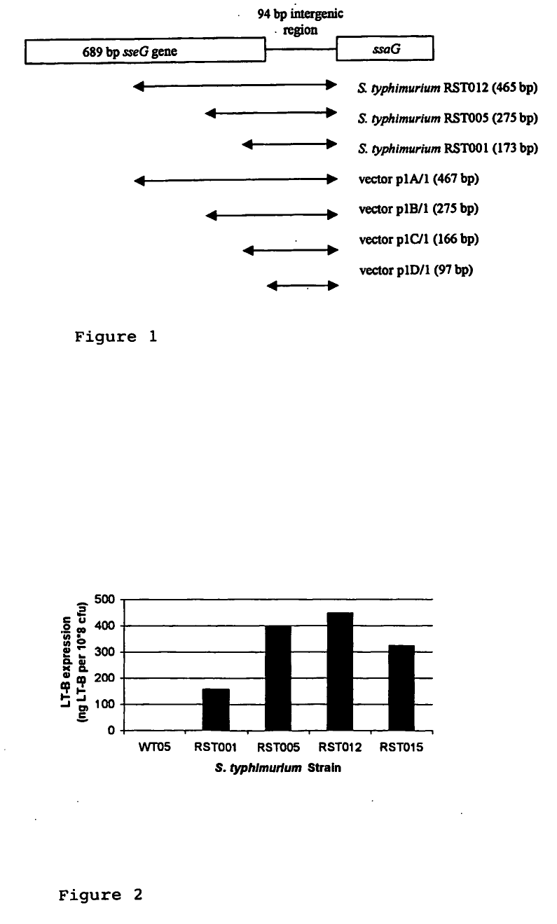

Table 1 summarises the list of strains used in this study. The associated ssaG promoter regions present in each strain and vector generated for use in this study are shown in FIG. 1.

Analysis of the open reading frames within the ssaG promoter region was carried out. The sseG gene lies upstream of the ssaG gene in S. typhimurium and the C-terminal portion of this protein is encoded within the ssaG promoter region shown in FIG. 1. There is a 94 bp intergenic region after the stop codon for the sseG gene and before the start codon of the ssaG gene. The intergenic region extends from base 414 to 506 of SEQ ID NO. 1, and is illustrated in FIG. 1. Therefore the 173, 275 and 465 bp ssaG promoter regions cloned into S. typhimurium RST001, RST005 and RST012 all contain part of the sseG coding region (however none of these constructs contain the entire sseG gene).

S. typhimurium harbouring defined mutations in ssaV and aroC are disclosed in WO00/68261. To supply the auxotrophic requirements of aroC⁻ mutants, all S. typhimurium aroC⁻ strains were routinely cultured at 37° C. in LB-aro broth (Luria Bertani broth supplemented with 10.0 μg/ml aminobenzoic acid, 40.0 μg/ml L-phenylalanine; 40.0 μg/ml L-tryptophan; 40.0 μg/ml tyrosine and 10 μg/ml 2,3-dihydroxybenzoic acid). Broth cultures were shaken at 180 rpm unless stated otherwise. 1.5% (w/v) agar was added to the above broth to generate solid media for the growth of aroC^{—} strains. S. typhimurium strain SL1344 was cultured in standard LB broth and the presence of the GFP reporter vectors transformed into this strain selected by the addition of 100 μg/ml ampicillin to the growth medium.

TABLE 1

| Strain name | Description |
|---|---|
| Salmonella | Wild type Salmonella typhimurium |
| typhimurium TML | |
| S. typhimurium | Salmonella typhimurium (TML aroC ssaV) WT05. (Wild type S. typhimurium |
| WT05 | TML harbouring defined deletions in the aroC and ssaV genes.) |
| S. typhimurium | Salmonella typhimurium (TML aroC ssaV) WT05 harbouring a chromosomal |
| RST001 | insertion at the site of the aroC deletion consisting of a 173 bp ssaG promoter |
| | region fused to the eltB gene |
| S. typhimurium | Salmonella typhimurium (TML aroC ssaV) WT05 harbouring a chromosomal |
| RST005 | insertion at the site of the aroC deletion consisting of a 275 bp ssaG promoter |
| | region fused to the eltB gene |
| S. typhimurium | Salmonella typhimurium (TML aroC ssaV) WT05 harbouring a chromosomal |
| RST012 | insertion at the site of the aroC deletion consisting of a 465 bp ssaG promoter |
| | region fused to the eltB gene |
| S. typhimurium | Salmonella typhimurium (TML aroC ssaV) WT05 harbouring a chromosomal |
| RST015 | insertion at the site of the aroC deletion consisting of the pagC promoter |
| | fragment fused to the eltB gene |
| S. typhimurium | Salmonella typhimurium strain SL1344. Used as host for pJKD10-based GFP |
| SL1344 | reporter vectors. |
| S. typhimurium | Salmonella typhimurium SL1344 harbouring vector p1A/1 (467 bp ssaG |
| BA267 | promoter region cloned into GFP reporter vector) |
| S. typhimurium | Salmonella typhimurium SL1344 harbouring vector p1B/1 (275 bp ssaG |
| BA269 | promoter region cloned into GFP reporter vector) |
| S. typhimurium | Salmonella typhimurium SL1344 harbouring vector p1C/1 (166 bp ssaG |
| BA271 | promoter region cloned into GFP reporter vector) |
| S. typhimurium | Salmonella typhimurium SL1344 harbouring vector p1D/1 (97 bp ssaG |
| BA273 | promoter region cloned into GFP reporter vector) |
| S. typhimurium | Salmonella typhimurium SL1344 harbouring vector pJKD10 (promoterless - |
| BA275 | GFP reporter vector) |

TABLE 2
Oligonucleotides used in this study

Restriction

| | | sites |
|---|---|---|
| Oligonucleotide | Oligonucleotide sequence (5′-3′) | incorporated |
| name | (5′ restriction sites are shown underlined) | into sequence |

| | | |
|---|---|---|
| 450SGF | GGATTGGCCTCGAGATTGCCATCGCGGATGTC | XhoI |
| 300SGF | GTAATGACTCGAGCATACTGGAGTGGTAGTT | XhoI |
| 150SGF | TCGGTATGGCTCGAGTGGCAATGACCGGTA | XhoI |
| SGR | AATATCCATATGGCTTTTCCTTAAAATAAA | NdeI |
| PAGCF | AGTTAACCACTCGAGATAATAATGGGTTTT | XhoI |
| PAGCR | AATAATATTTTTCATATGAACTCCTTAATACTA | NdeI |
| DESTM1 | CCTGGCAGGGATTGGGCATGCTATTGCCATCGCGGATGTCGCCT | SphI |
| DESTM2 | GACGGTAATGACGCATGCATACTGGAGTGGTAGTTTGGGACTA | SphI |
| DESTM3 | TATGGATGGGATGGCATGCACCGGTATGCAGGTCAGCAGCCCAT | SphI |
| DESTM4 | CCAGAACAACGTGCATGCGAGTAATCGTTTTCAGGTATATACCGG | SphI |
| DESTM5 | ACTAATTGTGCAATATGCATGCTGCTTTTCCTTAAAA | SphI |
| LTB/forward | TTCGGGATGACATATGAATAAAGTAAAATTT | NdeI |
| LTB/reverse | ATTAGACATGCTCCTAGGCTAGTCTAGTTTTCCATACTGATTGC | AvrII |

Strain Construction Methods

S. typhimurium RST001, RST005, RST015 and RST012 were all derived from the attenuated S. typhimurium strain WT05, which contains defined deletions in the aroC and ssaV genes of wild-type S. typhimurium TML. Construction of WT05 is described in WO00/68261. RST001, RST005 and RST012 contain promoter:gene fusions that have been integrated in the S. typhimurium WT05 chromosome at the site of the attenuating aroC deletion. In these three strains, the promoter gene fusions consist of a variable length of the ssaG promoter region fused to the eltB gene, which expresses the Escherichia coli heat labile toxin B subunit (LT-B). The ssaG promoter region corresponds to the region of DNA located immediately upstream of the ssaG gene in S. typhimurium and in this study the ssaG promoter region has been derived from S. typhimurium strain TML.

In S. typhimurium RST001, RST005 and RST012, the expression of LT-B is under the control of ssaG promoter regions of 173 bp, 275 bp and 465 bp respectively. To generate these promoters, three different primer pairs were used to PCR amplify these regions from the chromosomal DNA of S. typhimurium TML. Oligonucleotide primer pairs 450SGF/SGR, 300SGF/SGR, and 150SGF/SGR were used generate the 465, 275 and 173 bp fragments, respectively.

The primers resulted in the incorporation of 5′XhoI and 3′NdeI sites into each DNA amplicon. The eltB gene was PCR amplified from E. coli 078:H11 (American Type Culture Collection strain number 35401) using primers LTB/forward and LTB/reverse. The eltB amplicon, which contains a 5′ NdeI site and a 3′AvrII site, was subsequently ligated downstream of the 3′ NdeI sites present in the three ssaG promoter amplicons and the ssaG-eltB fusions cloned into the pBluescript II KS (+) cloning vector (Stratagene) modified to contain both XhoI and AvrII sites in its multiple cloning region. The ssaG-eltB fusions were then excised from pBluescript by digestion with XhoI and AvrII and inserted into the XhoI and AvrII sites present at the site of the aroC deletion in plasmid pMMIAC8. Plasmid pMMIAC8 is a pUC18 based vector, which contains a 4.8 kb HindIII fragment derived from S. typhimurium TML. The 4.8 kb insert harbours the S. typhimurium aroC gene into which has been engineered a defined 0.6 kb deletion. XhoI and AvrII restriction sites have been introduced at the 5′ and 3′ sites respectively of the deletion, and there is approximately 3 kb of upstream DNA and 1.7 kb of downstream DNA flanking the aroC gene. The nucleotide sequence of the constructs was confirmed by double strand nucleotide sequencing to ensure that the PCR steps had not introduced any errors.

Following insertion of the ssaG-eltB fusions into pMMIAC8, the resulting HindIII fragments were excised and cloned into the SmaI site in the suicide vector pCVD442. pCVD442 was selected for use as the suicide vector to deliver the modified DNA as it has previously been used to introduce defined deletions into the chromosome of Gram-negative bacteria. The resulting suicide constructs were then electroporated into S. typhimurium WT05. Resolution of the pCVD442 plasmid sequence and the original copy of the aroC deletion present in WT05 resulted in clones that contained the ssaG-eltB fusion inserted into the chromosome at the aroC deletion site. The integrity of the ssaG-eltB fusion was confirmed in these strains by Southern blotting and PCR. S. typhimurium RST015 was generated in a similar fashion to RST001, RST005 and RST012 except that the pagC promoter region was PCR amplified from S. typhimurium TML DNA, in place of the ssaG promoter using oligonucleotide primers PAGCF and PAGCR. The pagC-eltB fusion was inserted into pMMIAC8 and then transferred into the suicide vector pCVD442, in preparation for insertion into the S. typhimurium WT05 chromosome at the site of the aroC deletion.

Measurement of LT-B Expressed in S. typhimurium Strains Infecting the Human Macrophage-Like Cell Line, U397.

This method was used to assess the levels of LT-B expression from the ssaG and pagC promoters in S. typhimurium strains RST0001 RST005, RST012 and RST015. Bacteria were allowed to invade a U937 cell culture monolayer for 60 minutes, after which time any external bacteria were killed using gentamycin. At 24 hours post-infection, cells were lysed and the amount of LT-B expression determined in the GM-1 capture ELISA. The ELISA assay exploits the binding of LT-B to its cognate receptor protein, monosialo-ganglioside (GM-1). Flat-bottomed, 96-well Immulon 4 plates were coated overnight at 37° C. with 50 μls per well of GM-1 coating buffer (0.5 μg/ml of GM-1 (Sigma E-8015) dissolved in 50 mM glycine, 100 mM NaCl, 0.2 mM EDTA, 50 mM NaF, 0.1% (w/v) deoxycholate). The plates were then washed three times with PBST. Each well was then blocked (200 μl/well) with 3% (w/v) BSA in PBS for 1 hour at 37° C. with constant shaking. The plate was washed as above. 50-100 μl of bacterial lysate samples for GM-1 capture ELISA analysis were then added to wells and incubated for 1 hour at 37° C. with constant shaking. The plates were washed with PBST and 50 μl of 0.6 μg/ml Goat anti-LT (Biogenesis 4330-1104) dissolved in PBST plus 1% (w/v) BSA added to each well. Plates were incubated at 37° C. for 1 hour with constant shaking and then washed in PBST. Bound Goat anti-LT was detected by the addition of 50 μl/well of a mixture of biotinylated Rabbit anti-Goat Ig (Dako, E0466) and streptavidin peroxidase (Dako P0397), both diluted 1 in 2,500 in PBST plus 1% (w/v) BSA. Following incubation at 37° C. for 1 hour with constant shaking, the plates were washed in PBST and 100 μl/well of SigmaFast OPD detection reagent (Sigma, P-9187) added, dissolved according to the manufacturer's instructions. Plates were incubated for 10 minutes at room temperature and the OD₄₅₀ₙₘ recorded. Concentrations of LT-B in test samples were calculated from a standard curve prepared using purified LT (ICN Biomedicals, 151074) dissolved in PBST. LT-B concentrations were expressed as ng/ml per 10⁸ cfu.

U937 cells were selected as they are a macrophage-like cell-line, and macrophages are the in vivo site of replication for Salmonella strains. U937 cells were grown in 150 cm² tissue culture flasks containing 100 ml CRPMI (RPMI medium supplemented with 2 mM glycine, 10% (v/v) foetal calf serum (FCS), 100 units/ml penicillin, 100 μg/ml streptomycin). After 3-4 days growth at 37° C. in the presence of 5% CO₂, the cells were harvested and resuspended in cRPMI to give 3.0×10⁵ viable cells/ml. The cells were differentiated by adding 100 ng/ml phorbol myristate acetate (PMA). 24 ml aliquots of cells were then dispensed into 75 cm² tissue culture flasks, using 4 flasks per S. typhimurium strain to be tested. The flasks were incubated at 37° C. in 5% CO₂ for 96 hours or until a confluent monolayer was formed. 24 hours prior to the addition of S. typhimurium cells, the cRPMI medium was removed from the flasks, and 24 ml of PBS added to wash each flask. The PBS was removed by aspiration and replaced with 24 ml of RPMIg (RPMI supplemented with 2 mM glycine, 10% (v/v) FCS). For the preparation of the bacterial cultures used for the invasion studies, S. typhimurium strains were grown overnight in 20 ml of LB-aro broth, harvested and re-suspended in 20 ml of fresh LB-aro. The bacteria were opsonised by adding 75 μl of bacterial culture to 75 μl of human serum (human serum (minus IgA), Sigma, S5018). The samples were vortexed and incubated at room temperature for 20 minutes. 400 μl of RPMIg was then added to the cells. Immediately prior to the addition of bacteria, the tissue culture medium was removed from the U937 cells and replaced with 24 ml RPMIg (RPMIg supplemented with 10.0 μg/ml aminobenzoic acid, 40.0 μg/ml L-phenylalanine, 40.0 μg/ml L-tryptophan, 40.0 μg/ml tyrosine and 10 μg/ml 2,3-dihydroxybenzoic acid). 480 μl of opsonised cultures was added to each of the 4 flasks and the flasks incubated for 1 hour at 37° C. in 5% CO₂. The viable counts of the bacterial inocula were also recorded. In addition, the differentiated U937 cell count was determined by trypan blue exclusion staining. The culture medium was removed by aspiration and 24 ml of fresh RPMIg supplemented with 200 μg/ml gentamycin added to each flask and incubated at 37° C. in 5% CO₂ for 1 hour to kill extracellular bacteria. The tissue culture media was again removed and replaced with 24 ml RPMIg supplemented with 16 μg/ml gentamycin. At 24 hours post-infection, the culture medium was removed by aspiration and the cells washed with 24 ml of PBS. The U937 cells were lysed by adding 24 ml of PBS containing 1% (v/v) Triton X-100 to each flask and the samples incubated at room temperature for 20 minutes. The lysates were mixed thoroughly and harvested by centrifugation. The resulting pellets containing the S. typhimurium bacteria were resuspended in 100 μl PBS plus 0.05% (w/v) Tween-20 and incubated at room temperature for 10 minutes, with vortexing every 2 minutes. The lysates were stored at −20° C. until further use. 50 μl aliquots of lysates were used in the GM-1 capture ELISA described above to quantify LT-B expression.

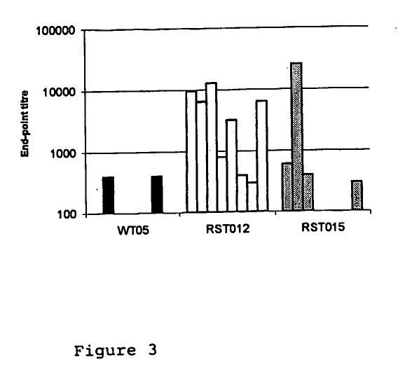

The results are shown in FIG. 2 and in each case the level of LT-B expression is expressed in ng LT-B per 10⁸ cfu's. The graph indicates that the 465, 275 and 173 bp fragments of the ssaG promoter all function to drive expression of proteins in S. typhimurium within the macrophage. Expression of LT-B in the S. typhimurium strain RST015, which harbours the pagC promoter fused to the eltB gene, has also been measured. LT-B expression from the pagC promoter is 28% less than in S. typhimurium RST012, supporting the improved efficacy of the 465 bp ssaG promoter under in vivo conditions.

In Vitro Study to Measure LT-B Expression in S. typhimurium Strains

Expression of LT-B by S. typhimurium from the in vivo inducible ssaG and pagC promoters was also compared in cells incubated in standard LB broth and in an intracellular salts medium (ISM) designed to approximate some of the environmental conditions experienced by Salmonella inside the host macrophage.

120 ml of S. typhimurium cultures were grown to late log phase, the cells harvested and resuspended in 20 ml of ISM (170 mM 2-[N-morpholino] ethanesulfonic acid (MES) pH 4.5, 0.5 mM MgSO₄ 1 μM CaCl₂, 6 mMK₂SO₄, 5 mM NH₄Cl₂, 5 mM NaCl, 0.4% (w/v) glucose, 2 μg/ml nicotinic acid). The cells were harvested again by centrifugation and resuspended in 4 ml of ISM. 250 ml of either ISM or LB broth were inoculated with 2.5 ml of resuspended culture and incubated at 37° C. under static conditions. At 0, 60, 90, 120 minutes and 20 hours post-inoculation, 50 ml was taken from each culture, the OD₆₀₀ recorded and 1 ml of culture removed for viable counting. The residual culture was harvested, and the pellet resuspended in 0.5 ml PBS supplemented with 0.05% (w/v) Tween-20. The samples were incubated at room temperature for 10 minutes to lyse the bacteria and then stored at −20° C. prior to ELISA analysis. To quantify LT-B expression, samples were diluted to approximately 1×10⁹ cfu/ml and 100 μl of this suspension used per well in the GM-1 capture ELISA. LT-B expression was measured over 20 hours and is expressed as ng LT-B produced per 10⁸ cfus. The overall expression levels of LT-B in ISM were lower than those observed in the macrophage study described above, implying that maximal induction from the promoters is not fully achieved in ISM and thus this medium does not fully replicate the conditions inside the macrophage.

After 120 minutes incubation in ISM, there was an 8-fold increase in LT-B expression in ISM in comparison to LB in the RST001 harbouring the 173 bp ssaG promoter region, and a 10-fold increase in RST012 harbouring the 465 bp ssaG promoter. In contrast, after 90 minutes, at which time LT-B expression is at its highest level in RST015 (pagC promoter), there is only a 2-fold increase in expression in ISM in comparison to LB. This is implies that although the pagC promoter may act as a more efficient promoter in the ISM medium, expression is less well regulated when compared to the ssaG promoter under standard in vitro (LB) growth conditions.

In Vivo Studies

Three in vivo studies were carried out to evaluate the levels of anti-LT-B IgG generated to S. typhimurium RST001, RST005, RST012 and RST015 in 6-8 week old BALB/c mice. In all studies, 8 mice were dosed per group and each mouse was immunized orally on day 0 with approximately 2-4×10¹⁰ cfus of the relevant strain, taken from an overnight culture in LB-aro. In study 1, mice were anaesthetized prior to immunization, whereas in studies 2 and 3 non-anaesthetized mice were used. Blood was sampled by tail bleeding at days 28 and by exsanguination at day 42. Sera were prepared by adding an equal volume of SeraSieve (Hughes and Hughes Ltd.) to the blood. Clotting of the blood was then allowed to proceed. at room temperature for 2 hours. The samples were centrifuged at 13000 rpm in a microcentrifuge for 10 minutes, and the supernatants (sera) collected and stored at −20° prior to ELISA analysis for anti-LT-B IgG (see method in section 2.5).

50 μl/well of GM-1 (Sigma, E-8015) dissolved to 30 μg/ml in 50 mM sodium carbonate buffer, pH 9.6, was used to coat wells of Dynex Immulon 4, 96-well flat-bottomed plates and incubated overnight at 4° C. The plates were then washed three times with PBST (phosphate buffered saline (PBS) containing 0.05% (w/v) Tween-20) and blocked by the addition of 200 μl/well of 3% (w/v) bovine serum albumin (BSA) in PBS. Following incubation for 1 hour at 37° C. with constant shaking, the plates were washed in PBST as described above. 50 μl of 1 μg/ml purified LT (ICN Biomedicals, 151074) dissolved in PBST was added to each well and the plate incubated for 1 hour at 37° C. with constant shaking before washing as described above. Two-fold serial dilutions of sera samples were prepared in PBST and 100 μl aliquots dispensed into wells. The plates were incubated at 37° C. for 1 hour with constant shaking and then washed with PBST. Horseradish peroxidase (HRP) conjugated Goat anti-Mouse IgG₁ (Southern Biotechnology, 1070-05) and HRP conjugated Goat anti-Mouse IgG₂ₐ (Southern Biotechnology, 1080-05) were mixed together at a 1/4000 dilution of each in PBST and 50 μl of the mixture added to each well. Following incubation at 37° C. for 1 hour with constant shaking, the plates were washed with PBST and the HRP conjugate detected by the addition of 100 μl per well of SigmaFast OPD detection reagent (Sigma, P-9187) prepared according to the manufacturers instructions. Following incubation at room temperature for 10 minutes, the reaction was stopped with the addition of 25 μl/well of 2 M sulphuric acid and the OD₄₉₂ₙₘ recorded. Results of this assay are expressed as end-point titres, which correspond to the last dilution of sera at which the OD₄₉₂ₙₘ is equal to the mean OD₄₉₂ₙₘ plus 3 times the standard deviation of the blank ells (blank well contain PBST in place of sera).

The end point titres of study 1 are presented in FIG. 3. 1 out of 8 mice immunized with the pagC construct, S. typhimurium RST015, responded with an end point titre of greater than 1000, whereas 5 out of 8 immunized with S. typhimurium RST012 responded with end point titres greater than 1000. None of the mice immunized with S. typhimurium WT05 produced an end point titre of greater than 1000. Analysis of the end point titres by the two-sample T-test was carried out to compare the results from RST012 and RST015. Immunisation with RST012 generates a significantly higher response than with RST015 (P=0.013).

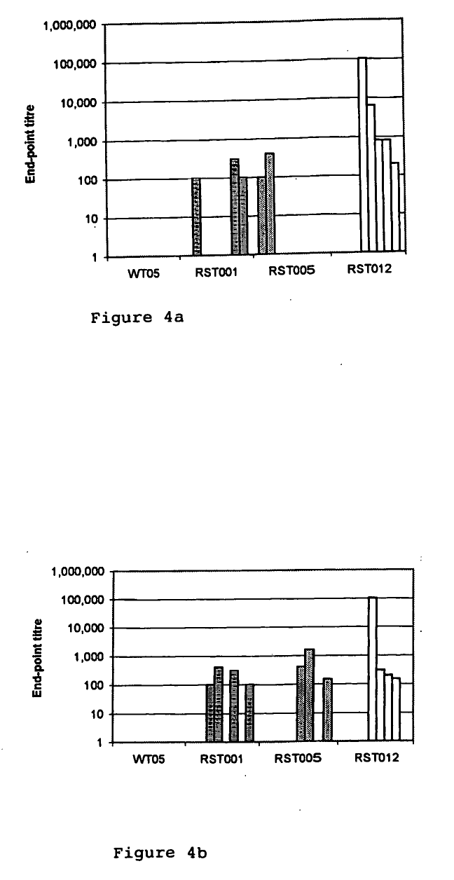

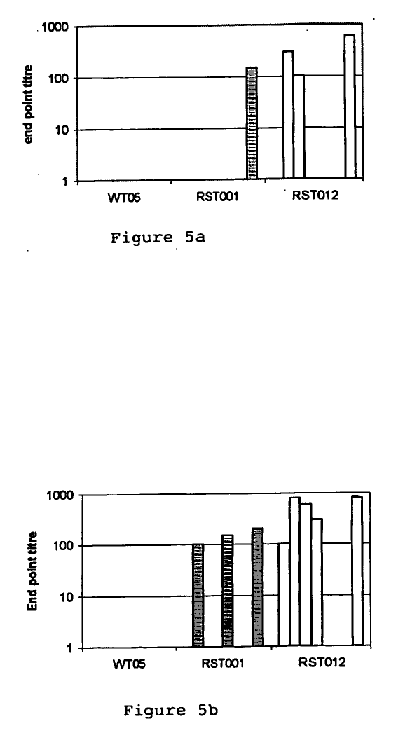

In study 2, the IgG responses to LT-B expressed in S. typhimurium from the 465, 275 and 173 bp ssaG promoter regions were compared at days 28 and 42 (see FIGS. 4a and 4b). In study 3, the responses resulting from the 465 and 173 bp ssaG promoter regions only were studied (FIGS. 5a and 5b). In contrast to study 1, studies 2 and 3 were carried out using non-anaesthetized mice. This explains the lower overall responses observed in studies 2 and 3 in comparison to study 1, as the use of anaesthetic during immunization has been found to generally augment immune responses in mice. Immune responses to LT-B were detected within all groups of mice receiving S. typhimurium strains harbouring the three variable ssaG promoter regions. No immune response to LT-B was detected in mice receiving the S. typhimurium WT05 control strain.

Construction of Green Fluorescent Protein (GFP) Reporter Vectors p1A/1, p1B/1, p1C/1 and p1D/1.

Table 3 shows the vectors used in this study.

TABLE 3

| Vector | |
|---|---|
| name | Description |
| p1A/1 | 467 bp ssaG promoter region cloned into GFP reporter vector |
| p1B/1 | 275 bp ssaG promoter region cloned into GFP reporter vector |
| p1C/1 | 166 bp ssaG promoter region cloned into GFP reporter vector |
| p1D/1 | 97 bp ssaG promoter region cloned into GFP reporter vector |
| PJKD10 | promoterless - GFP reporter vector |

Expression of the green fluorescent protein (GFP) under the control of various lengths of the ssaG promoter was also examined using the GFP reporter vector pJKD10. Promoters containing 467, 275, 166 and 97 bp of homologous DNA derived from upstream of ssaG were PCR amplified from S. typhimurium TML chromosomal DNA using the following pairs of oligonucleotides: DESTM1 and DESTM5 (467 bp); DESTM2 and DESTM5 (275 bp); DESTM3 and DESTM5 (166 bp), and DESTM4 and DESTM5 (97 bp) (see table 2). The amplicons were then digested with SphI and cloned into the SphI restriction site in pJKD10. pJKD10 is a 6.8 kb vector which contains the GFP reporter gene derived from pGFPmut3.1 (Clontech) cloned upstream of the lacZ reporter gene from pQF50. Two strong terminator sequences are also cloned upstream of the GFP gene to prevent read-through from the vector. The ssaG promoters were cloned immediately upstream of the GFP ‘ATG’ start codon and the correct orientation of the inserts confirmed by PCR. The resulting reporter vectors p1A/1 (467 bp ssaG promoter), p1B/1 (275 bp ssaG promoter), p1C/1 (166 bp ssaG promoter) and p1D/1 (97 bp ssaG promoter) were then transformed into S. typhimurium SL1344 by electroporation.

Fluorescence Activated Flow Cytometry (FACS) Analysis of GFP Expression from S. typhimurium Strains Infecting the Mouse Macrophage Cell Line J774A.1

This method was used to examine GFP expression in S. typhimurium strains transformed with reporter vectors by GFP fluorescent activated flow cytometry (FACS) analysis.

J774A.1 cells (ECACC#91051511) were cultured in DMEMg medium (Dulbeccos modified Eagles medium plus 1000 mg/L glucose (Sigma D5546) supplemented with 10% (v/v) heat-inactivated FCS (Sigma F9423), 2 mM L-glutamine (Sigma 7513) and Penicillin/Streptomycin (Sigma P0781) at a final concentration 10U/100 μg/ml). Prior to infection, J774A.1 cells were harvested and the cell count assessed by mixing cells 1:1 with trypan blue vital stain. The cells were diluted to 2×10⁵ viable cells/ml in DMEMg, and 0.5 ml volumes dispensed into each well of 24-well tissue culture plates. The plates were placed into a humidified CO₂ incubator at 37° C. for two days. On the day of the infection, the medium from each well containing J774A.1 cells was aspirated and the cells washed with three volumes of DMEMg. 0.5 ml of DMEMg was then added to each well. 100 μl aliquots of bacteria (10⁶ organisms per 100 μl) were added to the wells and to produce a multiplicity of infection (MOI) of 10 bacteria per J774A.1 cell. Infection was allowed to proceed for 30 minutes at 37° C. before the wells were washed three times with DMEMg. 0.5 ml of DMEMg supplemented with 50 μg/ml gentamycin to kill extracellular bacteria was added to each well and this stage in the experiment was recorded as time 0. At this and later time points, over a 24 hour period, selected wells were washed twice with phosphate buffered saline (PBS). The washed cells were fixed in 0.5 ml of 4% formaldehyde in PBS, for 20 minutes. After several washes in PBS, 0.5 ml of PBS was added to each well and the plates stored in the dark at 4° C. prior to FACs analysis. Wells were then washed with PBS, 0.5 ml PBS was added to each well and the cells resuspended by gentle scraping. The cells were recovered into tubes and GFP-generated fluorescence measured on the FL1 channel of a Becton Dickinson FACS Calibur flow cytometer utilising the air-cooled 488 nm argon laser. A total of 10,000 events were collected for each sample in duplicate and the cells analysed were those falling within the R1 region drawn around live cells. Data is expressed as the percentage cells expressing GFP greater than 10¹ Log10 fluorescence.

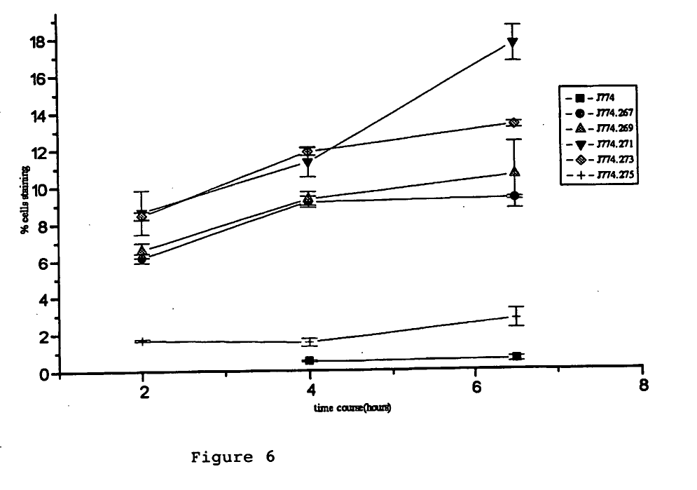

GFP expression was examined by FACS analysis at 2 hours, 4 hours and 6.5 hours post-invasion into the cell line (FIG. 6). Fluorescence induced by the various regions of the ssaG promoter were compared to two controls; uninfected J774A.1 cells and J774A.1 infected with S. typhimurium BA275 which contains the promoterless pJKD10 reporter vector. The levels of fluorescence (expressed as the % of cells staining) indicate that all the ssaG promoter fragments tested were capable of inducing GFP expression inside the macrophages in comparison to the controls. This data provides further evidence that ssaG promoter regions ranging from 467 bp to 97 bps can affect the in vivo inducible expression of proteins inside the macrophage.

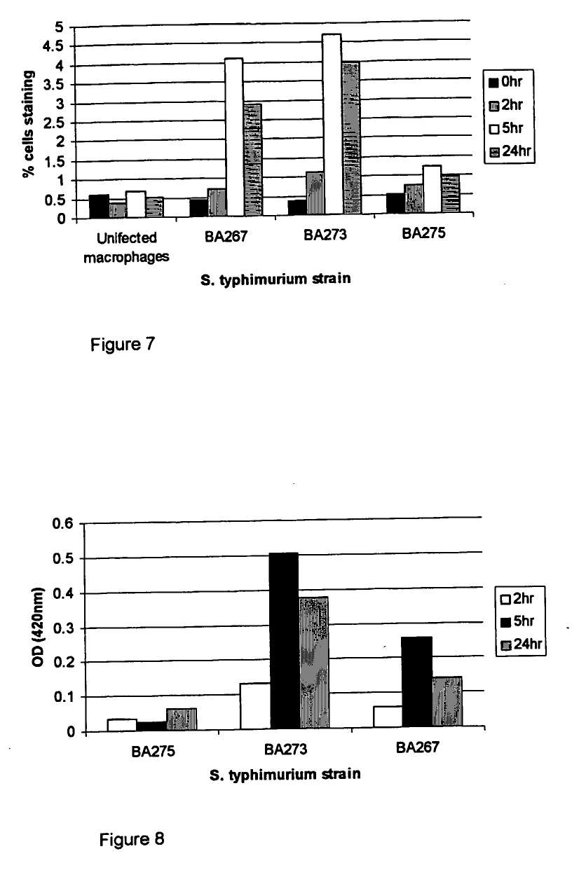

A further experiment was then performed in which the FACs analysis was extended to 24 hours (FIG. 7) and samples were also taken for analysis of LacZ expression (measured by colourimetric enzyme assay) (FIG. 8). The LacZ gene was cloned immediately down-stream of the GFP gene and permits an alternative and quantitative measure of gene expression. The experiment was performed exactly as described above except that at 0, 2, 5 and 24 hours post-infection, infected macrophages were also harvested by centrifugation and resuspended in 0.5 ml of sterile distilled water. The samples were then stored at −20° C. prior to LacZ analysis. To quantify LacZ expression, samples were thawed at room temperature and 144 μl of each sample placed into a well of a 96-well tissue culture plate (Costar 3590). 16 μl of 10×Z buffer (16.1 g Na₂HPO₄.7H₂O, 5.5 g NaH₂PO₄.H₂0, 0.75 g KCl, 0.246 g MgSO₄.7H₂O, 27 ml β-Mercapto-ethanol made up to 100 ml with distilled water, pH 7.0) was added. 32 μl of 4 mg/ml o-nitrophenyl-beta-D-galactoside solution (Sigma) dissolved in 1×Z buffer was added to each well and the plate incubated at 30° C. overnight. The reaction was stopped after 24 hours by the addition of 80 μl 1 M Na₂CO₃. The OD₄₂₀ₙₘ was measured for each well. The OD₄₂₀ₙₘ at the 0 hour time point was subtracted from the OD₄₂₀ₙₘ readings at the 2, 5 and 24 hour time points and the values plotted in the graph in FIG. 8.

The results show that expression of the two reporters is induced inside macrophages by both the 467 bp and 97 bp regions of the ssaG promoter in comparison to the promoterless control vector. The expression of the two reporter proteins correlates during the course of the experiment, with maximal expression of LacZ and GFP occurring at 5 hours post-invasion. Furthermore, the LacZ experiment shows that there remains a high level of expression of LacZ from both constructs at 24 hours post infection.

1. A construct comprising the ssaG promoter or a functional fragment thereof, operably linked to a polynucleotide heterologous to the ssaG gene.

2. A construct according to claim 1, wherein the promoter comprises at least the nucleotide sequence specified from nucleotide number 330 to 503 in SEQ ID NO. 1.

3. A construct according to claim 1 or claim 2, wherein the promoter comprises at least the nucleotide sequence specified from nucleotide number 229 to 503 in SEQ ID No. 1.

4. A construct according to any preceding claim, wherein the promoter comprises at least the nucleotide sequence from nucleotide number 39 to 503 of SEQ ID NO. 1.

5. A construct according to any preceding claim, wherein the heterologous polynucleotide encodes an antigen.

6. A construct according to any of claims 1 to 4, wherein the heterologous polynucleotide encodes a therapeutic protein, peptide or RNA.

7. An expression vector comprising a construct according to any preceding claim, for therapeutic use.

8. An integration vector capable of integrating into a host chromosome, comprising a construct according to any of claims 1 to 6.

9. A host cell comprising a product according to any preceding claim.

10. A host cell according to claim 9, wherein the cell is an animal cell.

11. A microorganism comprising a product according to any of claims 1 to 8.

12. A microorganism according to claim 11, which is a gram-negative bacterium.

13. A microorganism according to claim 11 or claim 12, which is Salmonella.

14. A microorganism according to claim 13, which is attenuated by the disruption of expression of the ssaV and aroC genes.

15. A Salmonella microorganism comprising a heterologous polynucleotide operably linked to the endogenous ssaG promoter.

16. A microorganism according to claim 15, wherein the heterologous polynucleotide is as defined in claim 5 or claim 6.

17. A microorganism according to any of claims 11 to 16, for therapy.

18. A vaccine composition comprising a microorganism according to any of claims 11 to 16.

19. Use of the ssaG promoter to promote expression of a polynucleotide heterologous to that of the ssaG gene and which encodes a therapeutic protein, peptide or RNA.

20. A method for the expression of a heterologous polynucleotide within a Salmonella microorganism, comprising integrating the polynucleotide into the Salmonella chromosome so that it is operably linked to the endogenous ssaG promoter.

21. A method for the production of a therapeutic product, comprising culturing a cell or microorganism according to any of claims 9 to 16 under conditions that permit the secretion of the therapeutic from the cell or microorganism, and isolating the therapeutic from the culture.


SEQUENCE LISTING (14 sequences)

SEQ ID NO: 1 - DNA, 506 bp, Salmonella typhimurium
gcgcgccgctcgtagccctggcagggattggccttgctattgccatcgcggatgtcgcctgtcttatctaccatcataaacatcatttgcctatggctcacgacagtataggcaatgccgttttttatattgctaattgtttcgccaatcaacgcaaaagtatggcgattgctaaagccgtctccctgggcggtagattagccttaaccgcgacggtaatgactcattcatactggagtggtagtttgggactacagcctcatttattagagcgtcttaatgatattacctatggactaatgagttttactcgcttcggtatggatgggatggcaatgaccggtatgcaggtcagcagcccattatatcgtttgctggctcaggtaacgccagaacaacgtgcgccggagtaatcgttttcaggtatataccggatgttcattgctttctaaattttgctatgttgccagtatccttacgatgtatttattttaaggaaaagcatt

SEQ ID NO: 2 - DNA, 32 bp, Artificial Sequence
  Description of Artificial Sequence Synthetic
ggattggcctcgagattgccatcgcggatgtc

SEQ ID NO: 3 - DNA, 31 bp, Artificial Sequence
  Description of Artificial Sequence Synthetic
gtaatgactcgagcatactggagtggtagtt

SEQ ID NO: 4 - DNA, 30 bp, Artificial Sequence
  Description of Artificial Sequence Synthetic
tcggtatggctcgagtggcaatgaccggta

SEQ ID NO: 5 - DNA, 30 bp, Artificial Sequence
  Description of Artificial Sequence Synthetic
aatatccatatggcttttccttaaaataaa

SEQ ID NO: 6 - DNA, 30 bp, Artificial Sequence
  Description of Artificial Sequence Synthetic
agttaaccactcgagataataatgggtttt

SEQ ID NO: 7 - DNA, 33 bp, Artificial Sequence
  Description of Artificial Sequence Synthetic
aataatatttttcatatgaactccttaatacta

SEQ ID NO: 8 - DNA, 44 bp, Artificial Sequence
  Description of Artificial Sequence Synthetic
cctggcagggattgggcatgctattgccatcgcggatgtcgcct

SEQ ID NO: 9 - DNA, 43 bp, Artificial Sequence
  Description of Artificial Sequence Synthetic
gacggtaatgacgcatgcatactggagtggtagtttgggacta

SEQ ID NO: 10 - DNA, 44 bp, Artificial Sequence
  Description of Artificial Sequence Synthetic
tatggatgggatggcatgcaccggtatgcaggtcagcagcccat

SEQ ID NO: 11 - DNA, 45 bp, Artificial Sequence
  Description of Artificial Sequence Synthetic
ccagaacaacgtgcatgcgagtaatcgttttcaggtatataccgg

SEQ ID NO: 12 - DNA, 37 bp, Artificial Sequence
  Description of Artificial Sequence Synthetic
actaattgtgcaatatgcatgctgcttttccttaaaa

SEQ ID NO: 13 - DNA, 31 bp, Artificial Sequence
  Description of Artificial Sequence Synthetic
ttcgggatgacatatgaataaagtaaaattt

SEQ ID NO: 14 - DNA, 44 bp, Artificial Sequence
  Description of Artificial Sequence Synthetic
attagacatgctcctaggctagtctagttttccatactgattgc